(19) United States
(12) Patent Application Publication
(10) Pub. No.: US 2024/0407669 A1
(43) Pub. Date: Dec. 12, 2024

(54) METHOD, APPARATUS AND RECORDING MEDIUM FOR ESTIMATING MOTOR FUNCTION INDEX VALUE, AND METHOD, APPARATUS AND RECORDING MEDIUM FOR GENERATING MOTOR FUNCTION INDEX VALUE ESTIMATION MODEL

(71) Applicants: PANASONIC HOLDINGS CORPORATION, Osaka (JP); POLARIS INC., Hyogo (JP)

(72) Inventors: Toshiaki TANAKA, Hyogo (JP); Maho SHIOTANI, Osaka (JP); Mika SAEKI, Tokyo (JP)

(21) Appl. No.: 18/808,336

(22) Filed: Aug. 19, 2024

Related U.S. Application Data
(63) Continuation of application No. PCT/JP2023/005097, filed on Feb. 15, 2023.

(30) Foreign Application Priority Data
Feb. 21, 2022 (JP) 2022-024860

(51) Int. Cl. A61B 5/11; A61B 5/00; G16H 10/60

(52) U.S. Cl. CPC A61B 5/1124; A61B 5/7264; G16H 10/60

(57) ABSTRACT

An information processing device acquires activity data about a target within a predetermined period of time and basic data including disease information about the target; extracts a plurality of feature amounts based on the activity data and the basic data having been acquired; inputs the plurality of feature amounts having been extracted into a learned motor function index value estimation model that is obtained by machine learning using, as training data, the plurality of feature amounts and actual measurement data of the motor function index value about each of a plurality of subjects to calculate the motor function index value about the target; and outputs the motor function index value having been calculated.

### FIELD OF INVENTION

The present disclosure relates to a method, a device, and a recording medium for estimating a motor function index value, and a method, a device, and a recording medium for generating a motor function index value estimation model.

### BACKGROUND ART

Non-Patent Literature 1 described below discloses a technique for estimating a TUG value of a target based on a measurement value of an acceleration sensor attached to the target.

In the technique for estimating the TUG value disclosed in Non-Patent Literature 1, the TUG value of the target is estimated based on only the measurement value of the acceleration sensor attached to the target, and thus the estimation accuracy is insufficient.

Non-Patent Literature 1: Saporito Salvatore et al., “Remote timed up and go evaluation from activities of daily living reveals changing mobility”, Physiological Measurement, 2019

### SUMMARY OF THE INVENTION

An object of the present disclosure is to obtain a method, a device, and a program for estimating a motor function index value that can estimate a motor function index value of a target, and a method, a device, and a program for generating a motor function index value estimation model.

A method for estimating a motor function index value according to one aspect of the present disclosure includes, by an information processing device, acquiring activity data about a target within a predetermined period of time and basic data including disease information about the target, extracting a plurality of feature amounts based on the activity data and the basic data having been acquired, inputting the plurality of feature amounts having been extracted into a learned motor function index value estimation model that is obtained by machine learning using, as training data, the plurality of feature amounts and actual measurement data of the motor function index value about each of a plurality of subjects to calculate the motor function index value about the target, and outputting the motor function index value having been calculated.

### BRIEF DESCRIPTION OF THE DRAWINGS

FIG. 1 is a diagram illustrating a simplified configuration of an information processing system according to an embodiment of the present disclosure.

FIG. 2 is a diagram illustrating a simplified configuration of a server device.

FIG. 3 is a flowchart showing generation processing of an estimation model executed by a processing unit of the server device in a learning phase.

FIG. 4 is a flowchart showing estimation processing of a TUG value executed by the processing unit of the server device in a use phase.

FIG. 5 is a graph showing estimation accuracy of the estimation model.

### DETAILED DESCRIPTION

### (Knowledge Underlying Present Disclosure)

In self-support nursing care facilities for elderly people and the like, deterioration in quality of nursing care services due to shortage of human resources and deterioration in productivity of nursing care staff, and non-uniformity in quality of nursing care services due to experience differences of nursing care staff and the like are problems on the site.

In order to improve and uniformize the quality of nursing care services, it is important to individually optimize the content of the nursing care service for each target, and in order to achieve the optimization, utilization of artificial intelligence is expected as a means for quantitatively evaluating the motor function of each target. By estimating the motor function of each target in advance based on data that can be measured and input at home or the like before entering or visiting a facility, the facility side can grasp the current state of the motor function of each target in advance, and as a result, it is possible to support individual optimization of the nursing care service. That is, the nursing care staff and the target and target's family share the recognition about the current state, the goal, the period of time or requirement required to achieve the goal, and the like, whereby selection and prioritization of services useful for the target himself/herself can be performed at an early stage before service start (contract). A situation in which there is a discrepancy in recognition between the nursing care staff and the target and target's family after the start of the service, and a situation in which an appropriate training method or care method is not determined or cannot be started even though a certain period of time has elapsed since entry are less likely to occur.

One of indices used to evaluate the motor function of the elderly and the like is a timed up and go (TUG) value. Non-Patent Literature 1 above discloses a technique including attaching an acceleration sensor to a target, remotely collecting a measurement value of the acceleration sensor within a predetermined period of time in which the target performs daily activities, and estimating a TUG value of the target based on the collected measurement value.

However, the technique for estimating the TUG value disclosed in Non-Patent Literature 1 includes estimation of the TUG value of the target based on only the measurement value of the acceleration sensor attached to the target, and thus the estimation accuracy is insufficient. In generation of the estimation model, users having a good motor function in which an actual measurement value of the TUG value is about 5 to 10 seconds are targeted as subjects, and a feature amount necessary for construction of an estimation model for users (TUG value of about 15 seconds or more) requiring self-support nursing care is not considered.

In order to solve such a problem, by performing machine learning using not only activity data of each subject but also disease information and the like with users in need of the self-support nursing care as a subject, the present inventor has made clear a feature amount necessary for generation of an estimation model for users in need of self-support nursing care, has found that it is possible to construct a highly accurate estimation model by using the feature amount, and has arrived at the present disclosure.

Next, each aspect of the present disclosure will be described.

A method for estimating a motor function index value according to a first aspect of the present disclosure includes: by an information processing device, acquiring activity data about a target within a predetermined period of time and basic data including disease information about the target; extracting a plurality of feature amounts based on the activity data and the basic data having been acquired; inputting the plurality of feature amounts having been extracted into a learned motor function index value estimation model that is obtained by machine learning using, as training data, the plurality of feature amounts and actual measurement data of the motor function index value about each of a plurality of subjects to calculate the motor function index value about the target; and outputting the motor function index value having been calculated.

According to the first aspect, the information processing device extracts the plurality of feature amounts based on activity data about the target within a predetermined period of time and basic data including disease information about the target, and inputs the plurality of feature amounts that are extracted to the learned motor function index value estimation model to calculate the motor function index value of the target. In this manner, use of not only activity data but also basic data including disease information enables the motor function index value of the target to be estimated with high accuracy.

In a method for estimating a motor function index value according to a second aspect of the present disclosure, in the first aspect, the motor function index value is a timed up and go (TUG) value.

According to the second aspect, the TUG value of the target can be estimated with high accuracy.

In a method for estimating a motor function index value according to a third aspect of the present disclosure, in the first or second aspect, the plurality of feature amounts include, as feature amounts based on the basic data, at least one of disease information, walking ability information, drug-taking information, meal intake status information, fluid intake status information, care level information, and physical information about the target.

According to the third aspect, the estimation accuracy of the motor function index value can be further improved.

In a method for estimating a motor function index value according to a fourth aspect of the present disclosure, in any one of the first to third aspects, the disease information includes a previous history of stroke about the target.

According to the fourth aspect, the estimation accuracy of the motor function index value can be further improved.

In a method for estimating a motor function index value according to a fifth aspect of the present disclosure, in any one of the first to fourth aspects, the plurality of feature amounts include, as feature amounts based on the activity data, at least one of walking information, exercise information, and sleeping hours information about the target.

According to the fifth aspect, the estimation accuracy of the motor function index value can be further improved.

In a method for estimating a motor function index value according to a sixth aspect of the present disclosure, in the fifth aspect, the walking information includes a maximum value and a standard deviation within the predetermined period of time about a total number of steps within a unit period of time, as well as a maximum value and a standard deviation within the predetermined period of time about a number of steps total value during continuous walking within the unit period of time.

According to the sixth aspect, the estimation accuracy of the motor function index value can be further improved.

A device for estimating a motor function index value according to a seventh aspect of the present disclosure includes: an acquisition unit that acquires activity data about a target within a predetermined period of time and basic data including disease information about the target; an extraction unit that extracts a plurality of feature amounts based on the activity data and the basic data having been acquired by the acquisition unit; a calculation unit that inputs the plurality of feature amounts having been extracted by the extraction unit into a learned motor function index value estimation model that is obtained by machine learning using, as training data, the plurality of feature amounts and actual measurement data of the motor function index value about each of a plurality of subjects to calculate the motor function index value about the target; and an output unit that outputs the motor function index value having been calculated by the calculation unit.

According to the seventh aspect, the extraction unit extracts the plurality of feature amounts based on activity data about the target within a predetermined period of time and basic data including disease information about the target, and the calculation unit inputs the plurality of feature amounts that are extracted by the extraction unit to the learned motor function index value estimation model to calculate the motor function index value of the target. In this manner, use of not only activity data but also basic data including disease information enables the motor function index value of the target to be estimated with high accuracy.

A program for estimating a motor function index value according to an eighth aspect of the present disclosure is a program for causing an information processing device to function as: an acquisition means configured to acquire activity data about a target within a predetermined period of time and basic data including disease information about the target; an extraction means configured to extract a plurality of feature amounts based on the activity data and the basic data having been acquired by the acquisition means; a calculation means configured to input the plurality of feature amounts having been extracted by the extraction means into a learned motor function index value estimation model that is obtained by machine learning using, as training data, the plurality of feature amounts and actual measurement data of the motor function index value about each of a plurality of subjects to calculate the motor function index value about the target; and an output means configured to output the motor function index value having been calculated by the calculation means.

According to the eighth aspect, the extraction means extracts the plurality of feature amounts based on activity data about the target within a predetermined period of time and basic data including disease information about the target, and the calculation means inputs the plurality of feature amounts that are extracted by the extraction means to the learned motor function index value estimation model to calculate the motor function index value of the target. In this manner, use of not only activity data but also basic data including disease information enables the motor function index value of the target to be estimated with high accuracy.

A method for generating a motor function index value estimation model according to a ninth aspect of the present disclosure includes: by an information processing device, acquiring activity data within a predetermined period of time, basic data including disease information, and actual measurement data of a motor function index value about each of a plurality of subjects; extracting a plurality of feature amounts based on the activity data and the basic data having been acquired; and generating a motor function index value estimation model by machine learning using, as training data, the plurality of feature amounts about each of the plurality of subjects and the actual measurement data.

According to the ninth aspect, the information processing device extracts the plurality of feature amounts based on the activity data of each subject within a predetermined period of time and the basic data including disease information about each subject, and generates a motor function index value estimation model by machine learning using, as training data, the plurality of feature amounts and actual measurement data about each subject. In this manner, use of not only the activity data but also the basic data including the disease information for extraction of the feature amount for machine learning enables a highly accurate motor function index value estimation model to be generated.

In a method for generating a motor function index value estimation model according to a tenth aspect of the present disclosure, in the ninth aspect, the motor function index value is a timed up and go (TUG) value.

According to the tenth aspect, a highly accurate TUG value estimation model can be generated.

In a method for generating a motor function index value estimation model according to an eleventh aspect of the present disclosure, in the ninth or tenth aspect, the plurality of feature amounts include, as feature amounts based on the basic data, at least one of disease information, walking ability information, drug-taking information, meal intake status information, fluid intake status information, care level information, and physical information about each of the plurality of subjects.

According to the eleventh aspect, the estimation accuracy of the motor function index value estimation model can be further improved.

In a method for generating a motor function index value estimation model according to a twelfth aspect of the present disclosure, in any one of the ninth to eleventh aspects, the disease information includes a previous history of stroke about each of the plurality of subjects.

According to the twelfth aspect, the estimation accuracy of the motor function index value estimation model can be further improved.

In a method for generating a motor function index value estimation model according to a thirteenth aspect of the present disclosure, in any one of the ninth to twelfth aspects, the plurality of feature amounts include, as feature amounts based on the activity data, at least one of walking information, exercise information, and sleeping hours information about each of the plurality of subjects.

According to the thirteenth aspect, the estimation accuracy of the motor function index value estimation model can be further improved.

In a method for generating a motor function index value estimation model according to a fourteenth aspect of the present disclosure, in the thirteenth aspect, the walking information includes a maximum value and a standard deviation within the predetermined period of time about a total number of steps within a unit period of time, as well as a maximum value and a standard deviation within the predetermined period of time about a number of steps total value during continuous walking within the unit period of time.

According to the fourteenth aspect, the estimation accuracy of the motor function index value estimation model can be further improved.

A device for generating a motor function index value estimation model according to a fifteenth aspect of the present disclosure includes: an acquisition unit that acquires activity data within a predetermined period of time, basic data including disease information, and actual measurement data of a motor function index value about each of a plurality of subjects; an extraction unit that extracts a plurality of feature amounts based on the activity data and the basic data having been acquired by the acquisition unit; and a generation unit that generates a motor function index value estimation model by machine learning using, as training data, the plurality of feature amounts about each of the plurality of subjects extracted by the extraction unit and the actual measurement data acquired by the acquisition unit.

According to the fifteenth aspect, the extraction unit extracts the plurality of feature amounts based on the activity data of each subject within a predetermined period of time and the basic data including disease information about each subject, and the generation unit generates a motor function index value estimation model by machine learning using, as training data, the plurality of feature amounts and actual measurement data about each subject. In this manner, use of not only the activity data but also the basic data including the disease information for extraction of the feature amount for machine learning enables a highly accurate motor function index value estimation model to be generated.

A computer-readable non-transitory recording medium recording a program for generating a motor function index value estimation model according to a sixteenth aspect of the present disclosure is a program for causing an information processing device to function as: an acquisition means that acquires activity data within a predetermined period of time, basic data including disease information, and actual measurement data of a motor function index value about each of a plurality of subjects; an extraction means that extracts a plurality of feature amounts based on the activity data and the basic data having been acquired by the acquisition means; and a generation means that generates a motor function index value estimation model by machine learning using, as training data, the plurality of feature amounts about each of the plurality of subjects extracted by the extraction means and the actual measurement data acquired by the acquisition means.

According to the sixteenth aspect, the extraction means extracts the plurality of feature amounts based on the activity data of each subject within a predetermined period of time and the basic data including disease information about each subject, and the generation means generates a motor function index value estimation model by machine learning using, as training data, the plurality of feature amounts and actual measurement data about each subject. In this manner, use of not only the activity data but also the basic data including the disease information for extraction of the feature amount for machine learning enables a highly accurate motor function index value estimation model to be generated.

An embodiment of the present disclosure will be described in detail below with reference to the drawings. Note that elements denoted by the same reference signs in different drawings represent the same or corresponding elements.

Note that each embodiment described below shows one specific example of the present disclosure. Numerical values, shapes, components, steps, order of steps, and the like shown in the following embodiment are merely examples, and are not intended to limit the present disclosure. A component that is not described in an independent claim representing the highest concept among components in the embodiment below is described as a discretionary component. In all the embodiments, respective items of content can be combined.

### EMBODIMENTS OF PRESENT DISCLOSURE

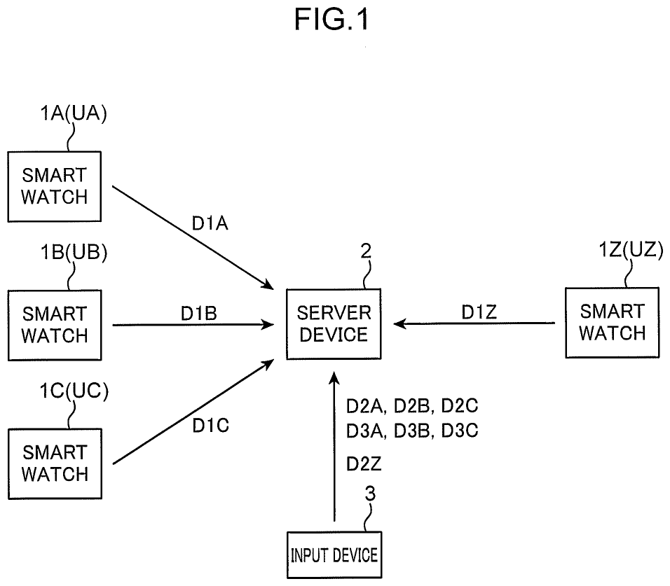

FIG. 1 is a diagram illustrating a simplified configuration of an information processing system according to an embodiment of the present disclosure. The information processing system includes a plurality of smartwatches 1, a server device 2, and an input device 3.

The smartwatch 1 includes smartwatches 1A to 1C worn by a plurality of subjects UA to UC and a smartwatch 1Z worn by a target UZ. The smartwatches 1A to 1C and 1Z are lent to the subjects UA to UC and the target UZ for a predetermined period of time such as two weeks. The subjects UA to UC and the target UZ continuously wear the smartwatches 1A to 1C and 1Z also while sleeping during the predetermined period of time.

The subjects UA to UC are users who provide activity data D1 (D1A to D1C) in order to generate an estimation model 31 of the motor function index value described later. The motor function index value is a timed up and go (TUG) value in the example of the present embodiment, but is not limited to this. For simplification of the drawing, FIG. 1 illustrates three subjects UA to UC, but the number of subjects is not limited to three as long as the number of subjects is plural. The target UZ is a user whose TUG value is to be estimated using the estimation model 31 having been learned. The subjects UA to UC and the target UZ are elderly persons of users in need of self-support nursing care, for example. The target UZ is a user who is before entering or visiting a self-support nursing care facility, for example.

The smartwatch 1 is an example of an activity tracker including an acceleration sensor and a heart rate sensor. The smartwatch 1 transmits, to the server device 2, the activity data D1 of the subject or the target measured by these sensors. For example, the smartwatch 1A transmits activity data D1A of the subject UA to the server device 2, and the smartwatch 1Z transmits activity data D1Z of the target UZ to the server device 2.

The server device 2 is an edge server on the premises, a cloud server out of the premises, or the like. The server device 2 can wirelessly communicate with the smartwatch 1 and the input device 3 via a discretionary communication network.

The input device 3 is a PC, a tablet, a smartphone, or the like with which input operation is possible by the user, a staff member of the self-support nursing care facility, or the like. The input device 3 transmits, to the server device 2, basic data D2 (D2A to D2C and D2Z) about the subjects UA to UC and the target UZ. The content of the basic data D2 will be described later. The input device 3 transmits, to the server device 2, actual measurement data D3A to D3C of the TUG value indicating the result of a TUG test conducted for each of the subjects UA to UC. The TUG test is a test for measuring the time required from the start of the standing-up motion until the subject again sits down in a series of motions in which the subject stands up from a state of being seated in a chair and starts walking, turns back at a target point 3 m ahead, and sits down again in the chair.

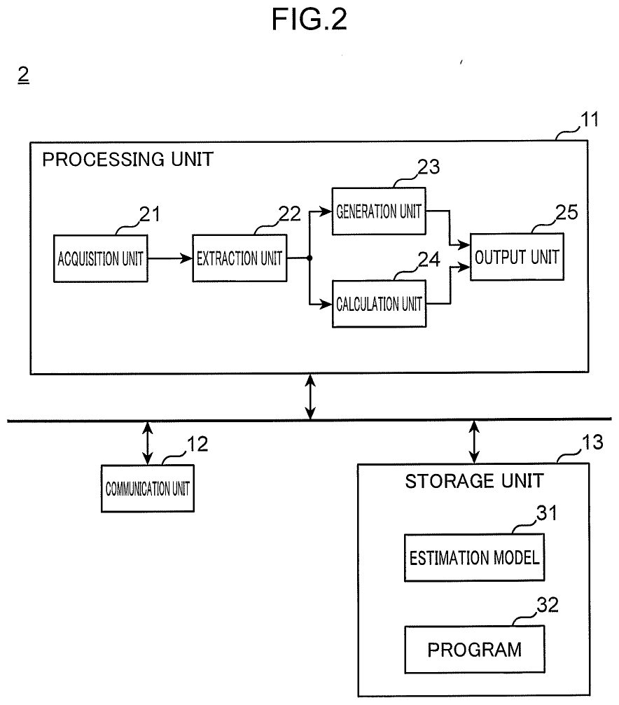

FIG. 2 is a diagram illustrating a simplified configuration of the server device 2. The server device 2 includes a processing unit 11, a communication unit 12, and a storage unit 13 connected to one another via a bus. The processing unit 11 is configured to include a processor such as a CPU. The communication unit 12 is configured to include a communication module compatible with a communication system with the smartwatch 1 and the input device 3. The storage unit 13 is configured to include an HDD, an SSD, or a semiconductor memory. The storage unit 13 stores an estimation model 31 having been learned. However, the estimation model 31 may be stored in an internal memory of not the storage unit 13 but the processing unit 11. The storage unit 13 stores a program 32.

As functions implemented by the processor executing the program 32 read from the storage unit 13, the processing unit 11 includes an acquisition unit 21, an extraction unit 22, a generation unit 23, a calculation unit 24, and an output unit 25. In other words, the program 32 is a program for generating the estimation model 31 for causing the server device 2 as an information processing device (device for generating estimation model) to function as the acquisition unit 21 (acquisition means), the extraction unit 22 (extraction means), the generation unit 23 (generation means), and the output unit 25 (output means) in a learning phase for generating the estimation model 31. The program 32 is a program for estimating a TUG value for causing the server device 2 as an information processing device (device for estimating TUG value) to function as the acquisition unit 21 (acquisition means), the extraction unit 22 (extraction means), the calculation unit 24 (calculation means), and the output unit 25 (output means) in a use phase of estimating the TUG value of the target UZ using the estimation model 31 having been learned.

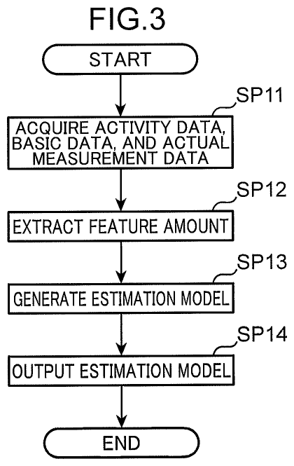

FIG. 3 is a flowchart showing the generation processing of the estimation model 31 executed by the processing unit 11 of the server device 2 in the learning phase.

First, in step SP11, the acquisition unit 21 acquires the activity data D1A to D1C of the subjects UA to UC collected within the predetermined period of time, the basic data D2A to D2C of the subjects UA to UC, and the actual measurement data D3A to D3C of the subjects UA to UC. At that time, the acquisition unit 21 may exclude, from the acquisition target, the activity data D1A to D1C of the first day and the final day of the predetermined period of time. By excluding the activity data D1A to D1C of the first day and the final day in which the wearing time of the smartwatch 1 is less than 24 hours, it is possible to eliminate abnormal values about the total number of steps and the like per day, and as a result, it is possible to improve the estimation accuracy of the estimation model 31.

The activity data D1 includes walking information, exercise information, and sleep information about each of the subjects UA to UC.

The walking information includes at least one of the following information.
a mean value, a maximum value, a standard deviation, a median value, and a minimum value within the predetermined period of time (e.g., two weeks) about the total number of steps within a unit period of time (e.g., one day)
a mean value, a maximum value, a standard deviation, a median value, and a minimum value within the predetermined period of time about the number of steps total value during continuous walking within the unit period of time (here, the continuous walking means walking without having a break for a predetermined time (e.g., one minute) or more)
a mean value, a maximum value, a standard deviation, a median value, and a minimum value within the predetermined period of time about a time total value during continuous walking within the unit period of time
a mean value, a maximum value, a standard deviation, a median value, and a minimum value within the predetermined period of time about a mean or a total exercise amount (METs) during continuous walking within the unit period of time
a mean value, a maximum value, a standard deviation, a median value, and a minimum value within the predetermined period of time about a mean or a total calorie consumption during continuous walking within the unit period of time
a mean value, a maximum value, a standard deviation, a median value, and a minimum value within the predetermined period of time about the mean cadence within the unit period of time (here, the cadence means the number of steps per minute)
a mean value, a maximum value, a standard deviation, a median value, and a minimum value within the predetermined period of time about a walking time during the longest continuous walking within the unit period of time
a mean value, a maximum value, a standard deviation, a median value, and a minimum value within the predetermined period of time about the number of steps during the longest continuous walking within the unit period of time
a mean value, a maximum value, a standard deviation, a median value, and a minimum value within the predetermined period of time about an exercise amount during the longest continuous walking within the unit period of time
a mean value, a maximum value, a standard deviation, a median value, and a minimum value within the predetermined period of time about the calorie consumption during the longest continuous walking within the unit period of time
sparsified walking information (e.g., “Low” if the total number of steps in the unit period of time is less than 1000 steps, “Medium” if 1000 steps or more and less than 5000 steps, and “High” if 5000 steps or more)

The exercise information includes at least one of the following information.
a mean value, a maximum value, a standard deviation, a median value, and a minimum value within the predetermined period of time about the total calorie consumption within the unit period of time
a mean value, a maximum value, a standard deviation, a median value, and a minimum value within the predetermined period of time about a mean heart rate during exercise within the unit period of time
a mean value, a maximum value, a standard deviation, a median value, and a minimum value within the predetermined period of time about a mean heart rate at rest within the unit period of time

The sleep information includes at least one of the following information.
a mean value, a maximum value, a standard deviation, a median value, and a minimum value within the predetermined period of time about night sleeping hours within the unit period of time (here, night means, for example, from 22:00 on the previous day to 7:00 on the day)
a mean value, a maximum value, a standard deviation, a median value, and a minimum value within the predetermined period of time about daytime sleeping hours within the unit period of time (here, daytime means, for example, from 7:00 to 15:00 on the day)
a mean value, a maximum value, a standard deviation, a median value, and a minimum value within the predetermined period of time about evening sleeping hours within the unit period of time (here, evening means, for example, from 15:00 to 22:00 on the day)
a mean value, a maximum value, a standard deviation, a median value, and a minimum value within the predetermined period of time about the number of times of awakening during the night within the unit period of time
a mean value, a maximum value, a standard deviation, a median value, and a minimum value within the predetermined period of time about the night awakening time total value within the unit period of time
a mean value, a maximum value, a standard deviation, a median value, and a minimum value within the predetermined period of time about a mean value of a sleep score (index representing sleep quality) within the unit period of time

The basic data D2 includes at least one of disease information, walking ability information, drug-taking information, meal intake status information, fluid intake status information, care level information, physical information, and daily life information about each of the subjects UA to UC.

The disease information includes at least one of the following information.
previous history of stroke, heart disease, orthopedic disease, neurogenic progression disease (Parkinson's disease, etc.), dementia, sleep disorder, mental disease, or cancer
presence or absence of paralysis, severity of paralysis, symptom type of dementia, presence or absence of restlessness of dementia, and presence or absence of higher brain function disorder
severity of each disease, presence or absence of contracture, severity of contracture, presence or absence of treatment of contracture, presence or absence of walking disorder, type of walking disorder, and severity of walking disorder
rehabilitation implementation content, rehabilitation implementation frequency, rehabilitation implementation time, rehabilitation implementation status in other facilities

The walking ability information includes at least one of the following information.
distance by which continuous walking is possible or practiced, time during which continuous walking is possible or practiced

The drug-taking information includes at least one of the following information.
presence or absence of a laxative, presence or absence of a soporific, a type of a soporific, presence or absence of a diabetes drug, a type of a diabetes drug, presence or absence of a diuretic, and other drug-taking information

The meal intake status information includes at least one of the following information.
meal form, calorie intake, meal content, and PFC balance

The fluid intake status information includes at least one of the following information.
fluid intake amount and fluid intake timing

The care level information includes at least one of the following information.
certification type of business target, in need of support, and in need of nursing care

The physical information includes at least one of the following information.
age, gender, height, weight, and BMI

The daily life information includes at least one of the following information.
whether or not to be capable of daily activities such as rising, sitting, rolling over, standing up, and standing
living condition information such as regularity of life rhythm and going-out frequency

Next, in step SP12, the extraction unit 22 extracts a plurality of feature amounts based on the activity data D1A to D1C and the basic data D2A to D2C acquired by the acquisition unit 21 by using a discretionary feature amount selection algorithm.

For example, the extraction unit 22 extracts at least one of walking information, exercise information, and sleeping hours information as the feature amount based on the activity data D1, and extracts at least one of disease information, walking ability information, drug-taking information, meal intake status information, fluid intake status information, care level information, and physical information as the feature amount based on the basic data D2.

In the example of the present embodiment, the extraction unit 22 uses the K-Best method as the feature amount selection algorithm. The extraction unit 22 extracts, as the feature amounts based on the activity data D1, a maximum value and a standard deviation within the predetermined period of time about the total number of steps within a unit period of time, and a maximum value and a standard deviation within the predetermined period of time about the number of steps total value during continuous walking within the unit period of time. The extraction unit 22 extracts a previous history of stroke as a feature amount based on the basic data D2.

As the feature amount selection algorithm, in place of the K-Best method, an RFE method, a K-means method, Forward-Selection method, a brute-force method, or the like may be used.

Next, in step SP13, the generation unit 23 uses, as training data, the plurality of feature amounts extracted by the extraction unit 22 and the actual measurement data D3A to D3C acquired by the acquisition unit 21, and generates the estimation model 31 of the TUG value by machine learning using a discretionary model algorithm.

In the example of the present embodiment, the generation unit 23 uses an SVR model as a model algorithm.

As the model algorithm, a linear multiple regression model, SVM, XGBoostRegression, a neural network, a hierarchical Bayesian model, or the like may be used.

Next, in step SP14, the output unit 25 outputs the estimation model 31 generated by the generation unit 23, and stores the estimation model 31 in the storage unit 13. However, in a case where the estimation model 31 is stored in the internal memory of not the storage unit 13 but the processing unit 11, the output processing of the estimation model 31 by the output unit 25 is omitted.

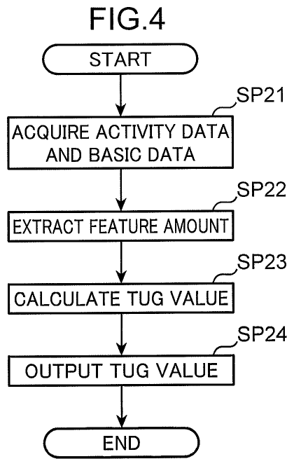

FIG. 4 is a flowchart showing the estimation processing of a TUG value executed by the processing unit 11 of the server device 2 in the use phase.

First, in step SP21, the acquisition unit 21 acquires the activity data D1Z of the target UZ collected within the predetermined period of time and the basic data D2Z of the target UZ. At that time, the acquisition unit 21 may exclude, from the acquisition target, the activity data D1Z of the first day and the final day of the predetermined period of time. By excluding the activity data D1Z of the first day and the final day in which the wearing time of the smartwatch 1 is less than 24 hours, it is possible to eliminate abnormal values about the total number of steps and the like per day, and as a result, it is possible to improve the estimation accuracy of the TUG value.

Next, in step SP22, the extraction unit 22 extracts a plurality of feature amounts based on the activity data D1Z and the basic data D2Z acquired by the acquisition unit 21 by using a discretionary feature amount selection algorithm.

For example, the extraction unit 22 extracts at least one of walking information, exercise information, and sleeping hours information as the feature amount based on the activity data D1Z, and extracts at least one of disease information, walking ability information, drug-taking information, meal intake status information, fluid intake status information, care level information, and physical information as the feature amount based on the basic data D2Z.

In the example of the present embodiment, the extraction unit 22 uses the K-Best method as the feature amount selection algorithm. The extraction unit 22 extracts, as the feature amounts based on the activity data D1Z, a maximum value and a standard deviation within the predetermined period of time about the total number of steps within a unit period of time, and a maximum value and a standard deviation within the predetermined period of time about the number of steps total value during continuous walking within the unit period of time. The extraction unit 22 extracts a previous history of stroke as a feature amount based on the basic data D2Z.

Next, in step SP23, the calculation unit 24 inputs the plurality of feature amounts extracted by the extraction unit 22 in step SP22 to the estimation model 31 having been learned generated in the learning phase to calculate the TUG value (estimation value) of the target UZ.

Next, in step SP24, the output unit 25 outputs the TUG value of the target UZ calculated by the calculation unit 24.

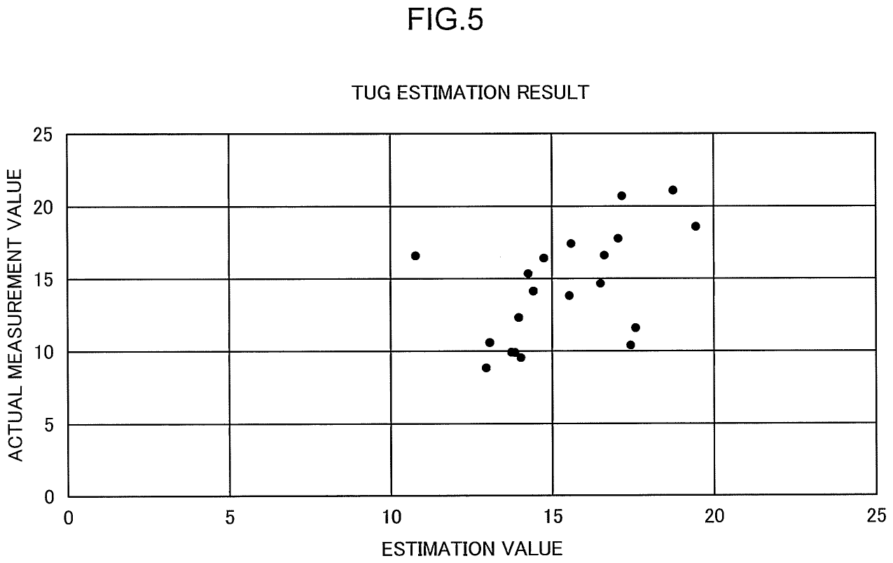

FIG. 5 is a graph showing estimation accuracy of the estimation model 31. As described above, targeting 19 subjects having an actual measurement value of the TUG value of about 8 to 22 seconds, a maximum value and a standard deviation within the predetermined period of time about the total number of steps within a unit period of time, a maximum value and a standard deviation within the predetermined period of time about the number of steps total value during continuous walking within the unit period of time, and a previous history of stroke were used as the feature amounts, thereby generating the estimation model 31. The vertical axis of the graph represents the actual measurement value of the TUG value, and the horizontal axis represents the estimation value of the TUG value using the estimation model 31. Although a large number of subjects having an actual measurement value of the TUG value of 15 seconds or more are included, the correlation coefficient exceeds 0.7, which suggests that high estimation accuracy is obtained.

According to the present embodiment, the estimation model 31 with high accuracy can be generated by using not only the activity data D1 but also the basic data D2 including disease information, and as a result, the TUG value of the target UZ can be estimated with high accuracy.

In the present embodiment, the activity data is acquired by the smartwatch, but the present disclosure is not limited to this. For example, the activity amount may be measured by a device including a sensor that can sense an activity amount, such as a gyro sensor, such as a smartphone or a pedometer.

The feature amount described above may further include sleep information, excretion information, physical condition information, inactivity level and period information, psychological/mental/motivation information, age/gender information, living alone/living together information, and external resource use information.

The disease information described above may further include a previous history, a symptom, cure, and treatment information.

The care level information described above may further include a care level, activities of daily living (ADL), and falling information such as the number of times/period of falling.

The meal intake status information described above may further include nutrition information such as an albumin value and denture information.

The basic data D2 may further include sleep information, excretion information, physical condition information, inactivity level and period information, psychological/mental/motivation information, age/gender information, living alone/living together information, and external resource use information.

The present disclosure is particularly useful for application to self-support nursing care AI for the purpose of improving and uniformizing the quality of nursing care services in self-support nursing care facilities.

1. A method for estimating a motor function index value, the method comprising:
by an information processing device,
acquiring activity data about a target within a predetermined period of time and basic data including disease information about the target;
extracting a plurality of feature amounts based on the activity data and the basic data having been acquired;
inputting the plurality of feature amounts having been extracted into a learned motor function index value estimation model that is obtained by machine learning using, as training data, the plurality of feature amounts and actual measurement data of the motor function index value about each of a plurality of subjects to calculate the motor function index value about the target; and
outputting the motor function index value having been calculated.

2. The method for estimating a motor function index value according to claim 1, wherein the motor function index value is a timed up and go (TUG) value.

3. The method for estimating a motor function index value according to claim 1, wherein the plurality of feature amounts include, as feature amounts based on the basic data, at least one of disease information, walking ability information, drug-taking information, meal intake status information, fluid intake status information, care level information, and physical information about the target.

4. The method for estimating a motor function index value according to claim 1, wherein the disease information includes a previous history of stroke about the target.

5. The method for estimating a motor function index value according to claim 1, wherein the plurality of feature amounts include, as feature amounts based on the activity data, at least one of walking information, exercise information, and sleeping hours information about the target.

6. The method for estimating a motor function index value according to claim 5, wherein the walking information includes a maximum value and a standard deviation within the predetermined period of time about a total number of steps within a unit period of time, as well as a maximum value and a standard deviation within the predetermined period of time about a number of steps total value during continuous walking within the unit period of time.

7. A device for estimating a motor function index value comprising:
an acquisition unit that acquires activity data about a target within a predetermined period of time and basic data including disease information about the target;
an extraction unit that extracts a plurality of feature amounts based on the activity data and the basic data having been acquired by the acquisition unit;
a calculation unit that inputs the plurality of feature amounts having been extracted by the extraction unit into a learned motor function index value estimation model that is obtained by machine learning using, as training data, the plurality of feature amounts and actual measurement data of the motor function index value about each of a plurality of subjects to calculate the motor function index value about the target; and
an output unit that outputs the motor function index value having been calculated by the calculation unit.

8. A computer-readable non-transitory recording medium recording a program for estimating a motor function index value, the program for causing an information processing device to perform a process comprising:
acquiring activity data about a target within a predetermined period of time and basic data including disease information about the target;
extracting a plurality of feature amounts based on the activity data and the basic data having been acquired;
inputting the plurality of feature amounts having been extracted into a learned motor function index value estimation model that is obtained by machine learning using, as training data, the plurality of feature amounts and actual measurement data of the motor function index value about each of a plurality of subjects to calculate the motor function index value about the target; and
outputting the motor function index value having been calculated.

9. A method for generating a motor function index value estimation model, the method comprising:
by an information processing device,
acquiring activity data within a predetermined period of time, basic data including disease information, and actual measurement data of a motor function index value about each of a plurality of subjects;
extracting a plurality of feature amounts based on the activity data and the basic data having been acquired; and
generating a motor function index value estimation model by machine learning using, as training data, the plurality of feature amounts about each of the plurality of subjects and the actual measurement data.

10. The method for generating a motor function index value estimation model according to claim 9, wherein the motor function index value is a timed up and go (TUG) value.

11. The method for generating a motor function index value estimation model according to claim 9, wherein the plurality of feature amounts include, as feature amounts based on the basic data, at least one of disease information, walking ability information, drug-taking information, meal intake status information, fluid intake status information, care level information, and physical information about each of the plurality of subjects.

12. The method for generating a motor function index value estimation model according to claim 9, wherein the disease information includes a previous history of stroke about each of the plurality of subjects.

13. The method for generating a motor function index value estimation model according to claim 9, wherein the plurality of feature amounts include, as feature amounts based on the activity data, at least one of walking information, exercise information, and sleeping hours information about each of the plurality of subjects.

14. The method for generating a motor function index value estimation model according to claim 13, wherein the walking information includes a maximum value and a standard deviation within the predetermined period of time about a total number of steps within a unit period of time, as well as a maximum value and a standard deviation within the predetermined period of time about a number of steps total value during continuous walking within the unit period of time.

15. A device for generating a motor function index value estimation model, the device comprising:
an acquisition unit that acquires activity data within a predetermined period of time, basic data including disease information, and actual measurement data of a motor function index value about each of a plurality of subjects;
an extraction unit that extracts a plurality of feature amounts based on the activity data and the basic data having been acquired by the acquisition unit; and
a generation unit that generates a motor function index value estimation model by machine learning using, as training data, the plurality of feature amounts about each of the plurality of subjects extracted by the extraction unit and the actual measurement data acquired by the acquisition unit.

16. A computer-readable non-transitory recording medium recording a program for generating a motor function index value estimation model, the program for causing an information processing device to perform a process comprising:
acquiring activity data within a predetermined period of time, basic data including disease information, and actual measurement data of a motor function index value about each of a plurality of subjects;
extracting a plurality of feature amounts based on the activity data and the basic data having been acquired; and
generating a motor function index value estimation model by machine learning using, as training data, the plurality of feature amounts about each of the plurality of subjects and the actual measurement data.
